(19) United States
(12) Patent Application Publication
(10) Pub. No.: US 2026/0144961 A1
(43) Pub. Date: May 28, 2026

(54) ACCESS DEVICE FOR USE WITH AN OCCLUSIVE MEMBER DELIVERY SYSTEM

(71) Applicant: BOSTON SCIENTIFIC SCIMED, INC., Maple Grove, MN (US)

(72) Inventors: Matthew Nelson Frost, New Brighton, MN (US); Andrea Goering, Blaine, MN (US); Matthew Maciej, Rogers, MN (US)

(73) Assignee: BOSTON SCIENTIFIC SCIMED, INC., Maple Grove, MN (US)

(21) Appl. No.: 19/396,600

(22) Filed: Nov. 21, 2025

Related U.S. Application Data
(60) Provisional application No. 63/723,891, filed on Nov. 22, 2024.

(51) Int. Cl. A61M 25/00; A61B 17/00; A61B 17/12; A61M 25/01; A61M 25/06

(52) U.S. Cl. CPC A61M 25/0045; A61M 25/0012; A61M 25/0026; A61M 25/005; A61M 25/0147; A61M 25/0662; A61B 2017/00526; A61B 2017/0084; A61B 17/12022; A61B 2017/1205; A61M 2025/0047; A61M 2205/0222; A61M 2207/00

(57) ABSTRACT

Catheter shafts are disclosed. An example catheter shaft may include a lubricious inner layer formed from a material that is free of per- and polyfluoroalkyl substances. A support layer may be disposed over the lubricious inner layer. The support layer may include a coil and a braid disposed over the coil. An outer layer may be extruded over the support layer.

### CROSS REFERENCE TO RELATED APPLICATIONS

The present application claims the benefit of U.S. Provisional Patent Application Ser. No. 63/723,891, filed Nov. 22, 2024, the disclosure of which is incorporated herein by reference.

### TECHNICAL FIELD

The present disclosure pertains to medical devices, and methods for manufacturing medical devices. More particularly, the present disclosure pertains to access devices for use with an occlusive member delivery system.

### BACKGROUND

A wide variety of medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices. A catheter shaft is disclosed. The catheter shaft comprises: a lubricious inner layer formed from a material that is free of per-and polyfluoroalkyl substances; a support layer disposed over the lubricious inner layer, the support layer including a coil and a braid disposed over the coil; and an outer layer extruded over the support layer.

Alternatively or additionally to any of the embodiments above, the lubricious inner layer is formed from a continuous layer of a single material.

Alternatively or additionally to any of the embodiments above, the lubricious inner layer includes ultra-high molecular weight polyethylene.

Alternatively or additionally to any of the embodiments above, the coil comprises a single filar coil.

Alternatively or additionally to any of the embodiments above, the coil comprises a ribbon coil.

Alternatively or additionally to any of the embodiments above, the coil defines an area of coverage that varies along a length of the support layer.

Alternatively or additionally to any of the embodiments above, the braid comprises a multi-strand braid.

Alternatively or additionally to any of the embodiments above, the braid varies in braid angle along a length of the support layer.

Alternatively or additionally to any of the embodiments above, the braid varies in braid spacing along a length of the support layer.

Alternatively or additionally to any of the embodiments above, the outer layer is formed from a continuous layer of a single material.

Alternatively or additionally to any of the embodiments above, the outer layer is free of per-and polyfluoroalkyl substances.

Alternatively or additionally to any of the embodiments above, further comprising a steering member coupled to the catheter shaft.

Alternatively or additionally to any of the embodiments above, further comprising a steering wire coupled to the steering member and extending proximally therefrom.

Alternatively or additionally to any of the embodiments above, further comprising a steering lumen, wherein the steering wire extends at least partially through the steering lumen.

A method for manufacturing a catheter shaft is disclosed. The method comprises: disposing a lubricious material as a continuous lubricious inner layer along a mandrel, the continuous lubricious inner layer being formed from a material that is free of per-and polyfluoroalkyl substances; disposing a support layer over the continuous lubricious inner layer, the support layer including a coil and a braid disposed over the coil; and disposing an outer material as a continuous outer layer over the support layer.

Alternatively or additionally to any of the embodiments above, the lubricious material includes ultra-high molecular weight polyethylene.

Alternatively or additionally to any of the embodiments above, disposing a support layer over the continuous lubricious inner layer includes varying an area of coverage of the coil along a length of the support layer.

Alternatively or additionally to any of the embodiments above, disposing a support layer over the continuous lubricious inner layer includes varying a braid angle of the braid along a length of the support layer.

Alternatively or additionally to any of the embodiments above, disposing a support layer over the continuous lubricious inner layer includes varying a braid spacing of the braid along a length of the support layer.

Alternatively or additionally to any of the embodiments above, disposing an outer material as a continuous outer layer over the support layer includes extruding the continuous outer layer over the support layer.

Alternatively or additionally to any of the embodiments above, the outer layer is free of per- and polyfluoroalkyl substances.

A catheter shaft is disclosed. The catheter shaft comprises: a continuous lubricious inner layer formed from a material that is free of per- and polyfluoroalkyl substances; a support layer disposed over the continuous lubricious inner layer, the support layer including a ribbon coil and a braid disposed over the ribbon coil; and a continuous outer layer extruded over the support layer.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a side view of an example delivery system.

FIG. 2 is a side view of an example delivery system.

FIG. 3 is a side view of a portion of an example access sheath.

FIG. 4 is a cross-sectional view taken through line 4-4 in FIG. 3.

FIG. 5 is a cross-sectional view of a portion of an example catheter shaft.

FIG. 6 is a cross-sectional view of a portion of an example catheter shaft.

FIG. 7 is a partially cut away view of a portion of an example catheter shaft.

FIG. 8 is a partially cut away view of a portion of an example catheter shaft.

FIG. 9 is a cross-sectional view of a portion of an example catheter shaft.

FIG. 10 is a cross-sectional view of a portion of an example catheter shaft.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

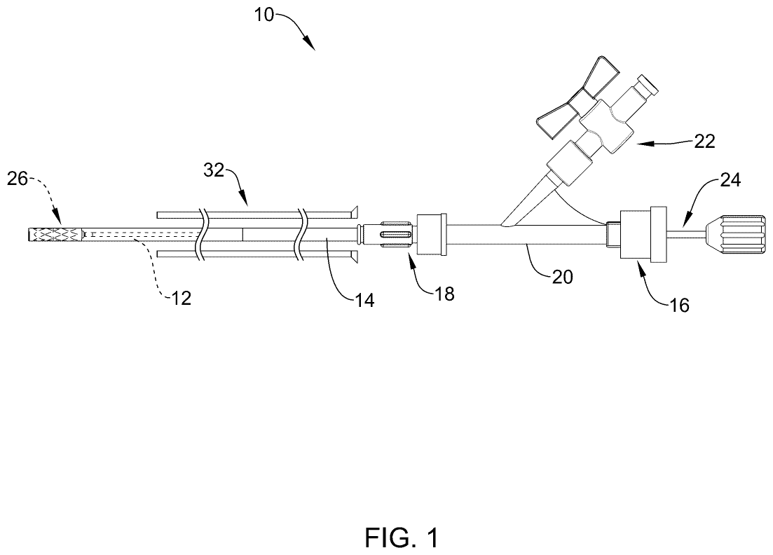

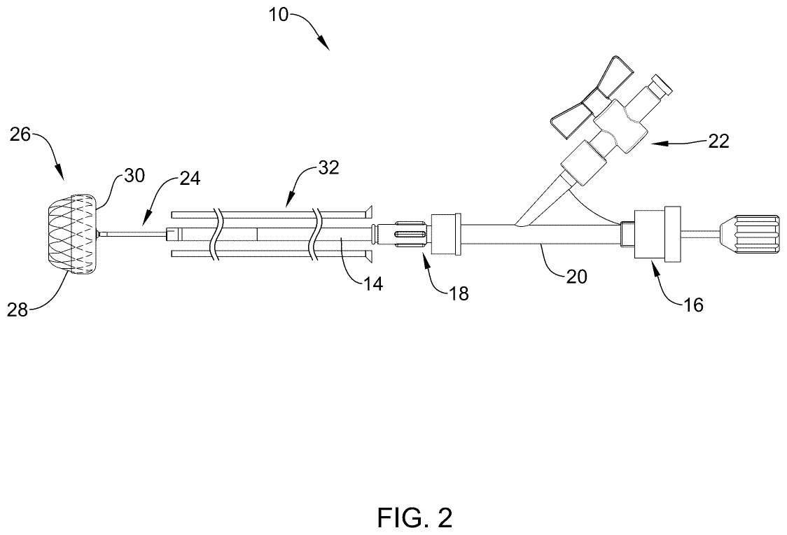

FIGS. 1-2 schematically illustrate selected components and/or arrangements of an occlusive implant system. It should be noted that in any given figure, some features of the occlusive implant system may not be shown, or may be shown schematically, for simplicity. Additional details regarding some of the components of the occlusive implant system may be illustrated in other figures in greater detail. The occlusive implant system may be used to deliver and/or deploy a variety of medical implants (e.g., a cardiovascular implant, an occlusive implant, etc.) to one or more locations within the anatomy, including but not limited to, in some instances, the heart and/or the left atrial appendage. In the interest of clarity, the following discussion refers to an occlusive implant, but other medical implants may be used and/or considered with the occlusive implant system. Example occlusive implants include WATCHMAN FLX™ and WATCHMAN FLX™ Pro from Boston Scientific.

The occlusive implant system may include a delivery system 10 including a delivery sheath 14 having a delivery lumen 12 extending proximally from a distal end of the delivery sheath 14. In one example, the delivery lumen 12 extends from a proximal opening to a distal opening of the delivery sheath 14. The delivery system 10 may include a proximal hub 16. In some instances, the delivery system may include a mid-hub 18. In some instances, the delivery system 10 may include a mid-shaft 20 extending from the proximal hub 16 to the mid-hub 18. In some instances, the delivery sheath 14 may extend distally from the mid-hub 18. Other configurations are also contemplated. In some instances, the delivery system 10 may include a side port 22. In some instances, the side port 22 may be in communication with the mid-shaft 20. Other configurations are also contemplated. In some instances, the delivery system 10 and/or the delivery lumen 12 may include a proximal segment (not shown) extending within and/or through the mid-hub 18, the mid-shaft 20, and the proximal hub 16. In some instances, the proximal segment may be in fluid communication with and/or may be an extension of the delivery lumen 12 of the delivery sheath 14. In some instances, the side port 22 may be in fluid communication with the proximal segment and/or the delivery lumen 12.

The occlusive implant system and/or the delivery system 10 may include a core member or core wire 24 slidably and/or rotatably disposed within the delivery lumen 12 (and the proximal segment, where present). The occlusive implant system may include an occlusive implant 26, which may be configured for implantation within a left atrial appendage, releasably engaged with and/or releasably attached to a distal end of the core wire 24. In at least some embodiments, the occlusive implant 26 may be a left atrial appendage closure device. In some instances, a proximal end of the core wire 24 may extend proximally of a proximal end of the delivery sheath 14 and/or the proximal opening of the delivery lumen 12 for manual manipulation by a clinician or practitioner. In at least some embodiments, the delivery sheath 14 may comprise and/or may be formed from a polymeric material. In some instances, the delivery sheath 14 may comprise and/or may be formed from a plurality of polymeric materials. In some instances, the delivery sheath may comprise and/or may be formed from a combination of metallic and polymeric materials. In some instances, the delivery sheath 14 may include a reinforcing element, such as a mesh, a coil, a braid, etc., formed therein, embedded therein, attached thereto, etc. along at least a portion of a length of the delivery sheath 14. Other configurations are also contemplated. Some suitable, but non-limiting, examples of materials for the occlusive implant system, the core wire 24, and/or the delivery sheath 14, etc., including but not limited to metallic materials, polymeric materials, etc., are discussed below.

The occlusive implant 26 may include an expandable framework 28 (e.g., FIG. 2) configured to shift between a delivery configuration (e.g., FIG. 1), such as when the occlusive implant 26 is disposed within the delivery lumen 12 proximate the distal opening and/or within a distal portion of the delivery lumen 12, and a deployed configuration (e.g., FIG. 2) when the occlusive implant 26 is unconstrained by the delivery sheath 14.

In some instances, the expandable framework 28 may comprise a plurality of interconnected struts. In some instances, the expandable framework 28 may be compliant or semi-compliant and may generally conform to and/or be configured to sealingly engage with the shape and/or geometry of the left atrial appendage in the deployed configuration.

In some instances, a proximal end of the expandable framework 28 may be configured to releasably attach, join, couple, engage, or otherwise connect to the distal end of the core wire 24 (e.g., FIG. 2). In some instances, the proximal end of the expandable framework 28 may include a proximal hub coupled and/or non-releasably attached thereto. In some instances, the proximal hub may be configured to and/or adapted to releasably couple with, join to, mate with, or otherwise engage a distal end of the core wire 24. Other means of releasably coupling and/or engaging the expandable framework 28 to the distal end of the core wire 24 are also contemplated.

In some instances, the occlusive implant 26 may include an occlusive element 30 (e.g., a membrane, a fabric, or a tissue element, etc.) connected to, disposed on, disposed over, disposed about, or covering at least a portion the expandable framework 28. In some instances, the occlusive element 30 may be connected to, disposed on, disposed over, disposed about, or cover at least a portion of an outer (or outwardly facing) surface of the expandable framework 28.

In some instances, the occlusive element 30 may be permeable or impermeable to blood and/or other fluids, such as water. In some instances, the occlusive element 30 may include a polymeric membrane, a metallic or polymeric mesh, a porous or semi-porous filter-like material, or other suitable construction. In some instances, the occlusive element 30 prevents thrombi (e.g., blood clots, etc.) from passing through the occlusive element 30 and out of the left atrial appendage into the blood stream. In some instances, the occlusive element 30 promotes endothelization after implantation, thereby effectively removing the target site (e.g., the left atrial appendage, etc.) from the patient's circulatory system. Some suitable, but non-limiting, examples of materials for the occlusive element 30 are discussed below.

In some instances, the expandable framework 28 and/or the plurality of interconnected struts may be integrally formed and/or cut from a unitary member. In some instances, the expandable framework 28 and/or the plurality of interconnected struts may be integrally formed and/or cut from a unitary tubular member and subsequently formed and/or heat set to a desired shape in the deployed configuration. In some instances, the expandable framework 28 and/or the plurality of interconnected struts may be integrally formed and/or cut from a unitary flat member or sheet, and then rolled or formed into a tubular structure and subsequently formed and/or heat set to the desired shape in the deployed configuration. Some exemplary means and/or methods of making and/or forming the expandable framework 28 include laser cutting, machining, punching, stamping, electro discharge machining (EDM), chemical dissolution, etc. Other means and/or methods are also contemplated.

In use, the delivery sheath 14 may be advanced and/or navigated to the left atrial appendage to deliver the occlusive implant 26 thereto. In one example, the delivery sheath 14 may be advanced and/or navigated to the left atrial appendage using and/or over a guidewire. For example, the delivery sheath 14 may be advanced to the patient's left atrium and the distal end disposed adjacent to the left atrial appendage with the occlusive implant 26 disposed therein in the delivery configuration. In some instances, the delivery sheath 14 may include steering capability. After the distal end of the delivery sheath 14 is disposed adjacent to and/or at the left atrial appendage, the core wire 24 may be advanced distally relative to the delivery sheath 14 to advance the occlusive implant 26 out of the delivery sheath 14, where the occlusive implant 26 may shift to the deployed configuration.

In some instances, the delivery system 10 may further comprise an access sheath 32. For example, the access sheath 32 may be advanced and/or navigated toward the left atrial appendage. In some instances, the access sheath 32 may include steering capability. During use, the delivery sheath 14 may be advanced within the access sheath 32 with the occlusive implant 26 disposed therein in the delivery configuration. After the distal end of the delivery sheath 14 is disposed adjacent to and/or at the distal end of the access sheath 32, the core wire 24 may be advanced distally relative to the delivery sheath 14 and/or the access sheath 32 to advance the occlusive implant 26 out of the delivery sheath 14 and the access sheath 32, where the occlusive implant 26 may shift to the deployed configuration. Disclosed herein are access sheaths that may, for example, be used in conjunction with an occlusive device and/or a delivery system for delivering an occlusive device/implant.

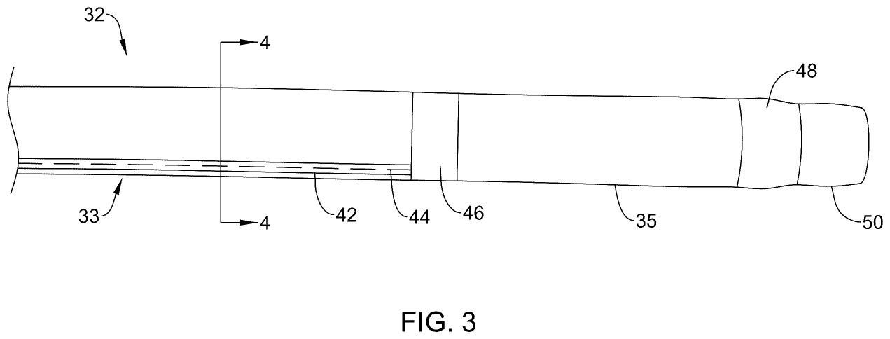

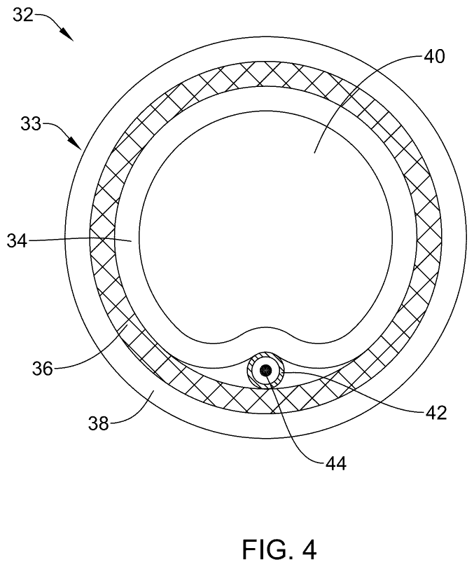

An example access sheath 32 is schematically depicted in FIGS. 3-4. The access sheath 32 includes a sheath or sheath body 33 having a distal end region 35. A lumen 40 may be defined through the sheath 33. A marker member 48 (e.g., a radiopaque marker formed from or including a radiopaque material) may be coupled to the sheath 33, for example adjacent to a distal end region or a tip 50 of the sheath 33. The form of the access sheath 32 may vary. For example, as shown in FIG. 4 the sheath 33 may be formed from a plurality of layers such as an inner layer or liner 34, a reinforcing member or layer 36, and an outer layer 38. The inner layer 34 may include a lubricious material such as polytetrafluoroethylene. Other materials including those disclosed herein may be utilized. The reinforcing layer 36 may include a braid, a coil, both a braid and a coil, and/or the like. The outer layer may include a polyether block amide. Other materials including those disclosed herein may be utilized.

A low friction tube 42 may be disposed within the lumen 40. The low friction tube 42 may resemble a small/miniature catheter shaft and may be disposed along the inner wall of the sheath 33. Alternatively, the low friction tube 42 may be integrated between the inner layer 34 and the reinforcement layer 36 of the access sheath 32. In some instances, the low friction tube 42 may be formed from a single layer of lubricious material or a number of layers. For example, the low friction tube 42 may include an inner lubricious layer (e.g., formed from polytetrafluoroethylene or the like), a reinforcing braid, and an outer layer formed from polyether block amide. Other materials including those disclosed herein may be utilized. In some instances, a plurality of low friction tubes 42 may be disposed within the lumen 40 and/or integrated between the inner layer 34 and the reinforcement layer 36. A steering wire 44 may be disposed within the low friction tube 42. As the name suggests, the steering wire 44 may be used to steer, bend, and/or otherwise deflect the sheath 33.

Shafts such as the delivery sheath 14, the access sheath 32, other catheter shafts and sheaths, and/or the like may be designed to include a number of structural features. For example, a catheter shaft may be manufactured to include a lubricious inner layer, a reinforcement layer, and an outer layer. In some instances, the lubricious inner layer may include per- and polyfluoroalkyl substances. It may be desirable to utilize materials that are free of per- and polyfluoroalkyl substances. In addition, the outer layer may vary along the length of the catheter shaft to vary one or more physical characteristics of the catheter shaft. For example, the outer layer may vary in material composition so as to provide differing flexibility characteristics to the catheter shaft along the length thereof. When varying the composition of the outer layer, a fair amount of scrap or waste material may be produced. It may be desirable to vary the physical characteristics of the catheter shaft in a manner that minimizes waste. Disclosed herein are catheter shafts and methods for manufacturing catheter shafts that, for example, utilize materials that are free of per- and polyfluoroalkyl substances, vary the physical characteristics of the catheter shaft in a manner that minimizes waste, and/or provide other desirable benefits. The features of the disclosed catheter shafts may be incorporated into a variety of different devices including delivery sheaths (e.g., similar to the delivery sheath 14), access sheaths (e.g., similar to the access sheath 32), other catheter shafts, and/or the like.

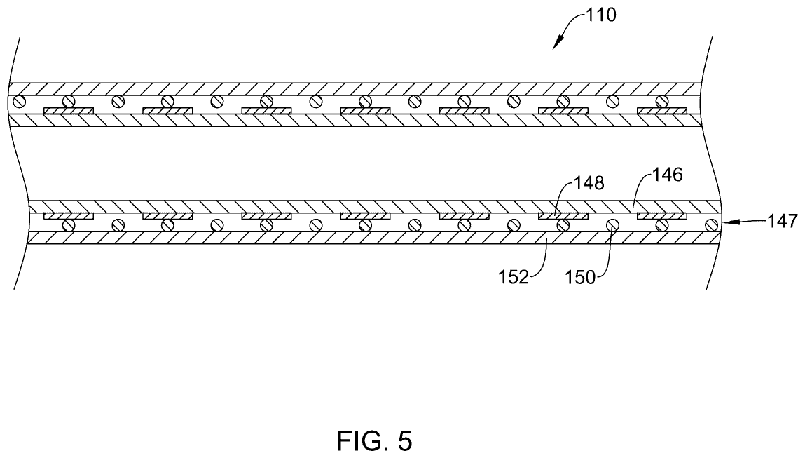

FIG. 5 illustrates an example catheter shaft 110. The catheter shaft 110 may be any one of a number of different shafts/sheaths including a delivery sheath (e.g., similar to the delivery sheath 14), an access sheath (e.g., similar to the access sheath 32), another type of catheter shaft, and/or the like. The catheter shaft 110 may include an inner layer 146, a reinforcing layer 147, and an outer layer 152. In at least some instances, the reinforcing layer 147 may include a coil member 148, a braid 150, or both (e.g., as depicted). In this example, the coil member 148 is disposed over/along the inner layer 146 and the braid 150 is disposed over/along the coil member 148. Other arrangements are contemplated including arrangements where the coil member 148 is disposed over the braid 150.

In some instances, the coil member 148 may be a single filar coil. Multi-filar coils are also contemplated. The coil member 148 may have a rectangular cross-section or ribbon shape. Other shapes including round or rounded shapes are contemplated. The coil member 148 may be formed form a metal (e.g., such as those listed herein), a polymer (e.g., nylon-12, polyetheretherketone, polyimide, and/or suitable materials such as those listed herein). The braid 150 may be a multi-strand braid. A variety of arrangements are contemplated for the braid 150.

In at least some instances, the inner layer 146 may be formed from a material that is free of per- and polyfluoroalkyl substances. For example, the inner layer 146 may be formed from ultra-high molecular weight polyethylene. Other materials may be utilized including thermoplastic materials and/or suitable materials disclosed herein. In at least some instances, the inner layer 146 may be a continuous layer formed from a singular material. The inner layer 146 may be formed, for example, by disposing (e.g., extruding, molding, etc.) a layer of material onto a mandrel (e.g., a disposable or dissolvable mandrel). After forming the inner layer 146, the reinforcing layer 147 may be disposed onto the inner layer 146. The outer layer 152 may be disposed onto (e.g., via extruding, molding, etc.) the reinforcing layer 147. In at least some instances, the outer layer 152 may be a continuous layer formed from a singular material. In at least some instances, the outer layer 152 may be formed from a material that is free of per- and polyfluoroalkyl substances. Other materials may be utilized including thermoplastic materials and/or suitable materials disclosed herein. The outer layer 152 may be formed from the same or a different material from the inner layer 146.

Forming the catheter shaft 110 in this manner may be described as being continuous or continuously formed. In some instances, continuously forming the catheter shaft 110 (and/or the layers thereof) may include a reel-to-reel process. Such processes may avoid heat shrink or other bonding steps that may otherwise be utilized when joining together different sections of material such as outer layer sections of different material composition. By avoiding heat shrink processes, materials such as per-and polyfluoroalkyl materials can be avoided. In other instances, different sections of the outer layer 152 (e.g., formed from different material) can be disposed along the reinforcing layer 147, for example by extrusion. Alternatively, sections of the outer layer 152 can be removed (e.g., via ablation) and a new section of material may be added. Additional outer layers or coating can also be disposed along the outer layer 152.

It can be appreciated that when the catheter shaft 110 is intended to be used as an access sheath (e.g., similar to the access sheath 32), other structures such as a low friction tube (e.g., similar to the low friction tube 42), a steering wire (e.g., similar to the steering wire 44), etc. may be incorporated into the catheter shaft 110. When the catheter shaft 110 is to be used as a different type of device, other structures may be incorporated such as electrode wires, electrode wire lumens, sensors (e.g., pressure, impedance, location, etc.), balloons, and/or the like.

In at least some instances, the variations in the physical properties of the catheter shaft 110 may be primarily based on, or substantially solely based on, variations in the reinforcing layer 147. For example, the reinforcing layer 147 may be varied along the length of the catheter shaft 110 in order to vary the flexibility of the catheter shaft 110. This may include varying the pitch, angle, and/or the like (e.g., along a length of the reinforcing layer 147 and/or the inner layer 146) of the coil member 148. Another way to assess/quantify variations in the coil member 148 may include variations in the amount of surface area coverage that the coil material of the coil member 148 presents along the catheter shaft 110 (e.g., the inner layer 146). For example, zero spacing between windings/revolutions of the coil member 148 would equate to 100% coverage. Larger spacing equates to lesser coverage and/or surface area. It can be appreciated that the coil member 148 may tend to be more flexible as the percent coverage decreases (e.g., coverage area is inversely proportional to flexibility). In at least some instances, the coil member 148 defines an area of coverage that varies along a length of the reinforcing layer 147. In some instances, a generally stiffer, more proximal section of the catheter shaft 110 may have a coil member 148 with an area of coverage in the range of about 65%-90% whereas a generally flexible, more distal section of the catheter shaft 110 may have a coil member 148 with an area of coverage in the range of about 10%-35%. The area of coverage range of the coil member 148 between the more proximal and more distal sections may be between that of the more proximal and distal sections (e.g., greater than about 10% to less than about 90%, or about 25%-75%, or about 35%-65%).

In some of these and in other instances, the braid 150 may vary in braid angle, PIC (per inch crossings) count, braid spacing, and/or the like. In at least some instances, the braid 150 may have a braid spacing, a braid angle, and/or the like along the length of the reinforcing layer 147. Another way to assess/quantify variations in the braid 150 may include variations in the amount of surface area that the braid material of the braid 150 covers the catheter shaft 110 (e.g., the inner layer 146). For example, zero spacing between windings/crossings of the braid 150 would equate to 100% coverage. Larger spacing equates to lesser coverage and/or surface area. In some instances, a generally stiffer, more proximal section of the catheter shaft 110 may have a braid 150 with a braid angle of about 45-50 degrees (e.g., corresponding to lower braid surface area coverage) whereas a generally flexible, more distal section of the catheter shaft 110 may have a braid 150 with a braid angle of about 55-70 degrees (e.g., corresponding to higher braid surface area coverage). The braid angle of the braid 150 between the more proximal and more distal sections may be between that of the more proximal and distal sections (e.g., greater than about 45 degrees to less than about 70 degrees, or about 48-60 degrees, or about 50-55 degrees).

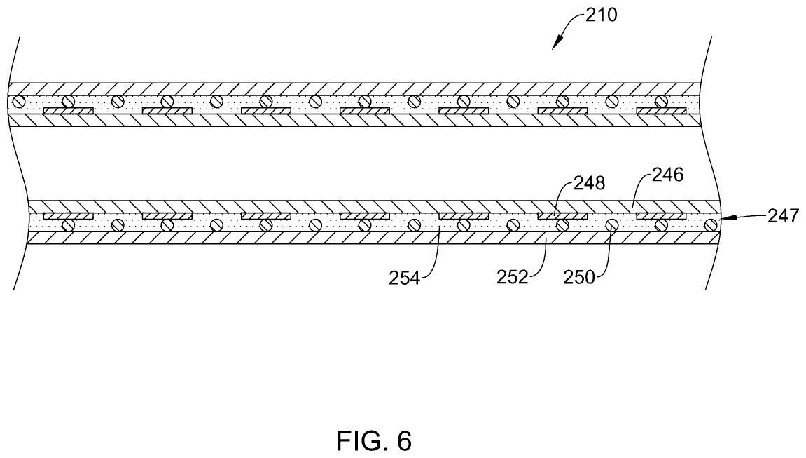

FIG. 6 illustrates another example catheter shaft 210 that is similar in form to other catheter shafts disclosed herein. The catheter shaft 210 may include an inner layer 246, a reinforcing layer 247, and an outer layer 252. In at least some instances, the reinforcing layer 247 may include a coil member 248, a braid 250, or both (e.g., as depicted). In this example, the material of the outer layer 252 and the inner layer 246 may be able to contact one another and form a blended layer 254 (e.g., formed as combination of the outer layer 252 and the inner layer 246) that extends through the reinforcing layer 247. This may help to form a bond between the outer layer 252 and the inner layer 246. In some instances, rather than forming a blended layer, the outer layer 252 and the inner layer 246 may remain distinct such that a detectable or discernable boundary still exists between the layers. In such instances, the outer layer 252 and the inner layer 246 may be formed from different materials that do not fully blend with one another.

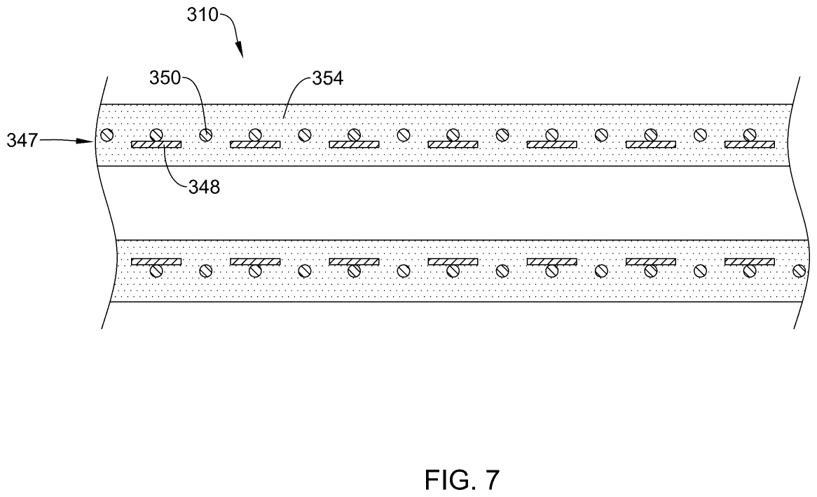

FIG. 7 illustrates another example catheter shaft 310 that is similar in form to other catheter shafts disclosed herein. The catheter shaft 310 may include a reinforcing layer 347 disposed within a continuous layer 354. In at least some instances, the reinforcing layer 347 may include a coil member 348, a braid 350, or both (e.g., as depicted). The continuous layer 354 may be understood to be a substantially homogenous layer that encapsulates the reinforcing layer 347. The continuous layer 354 may be include a singular material or a combination of materials.

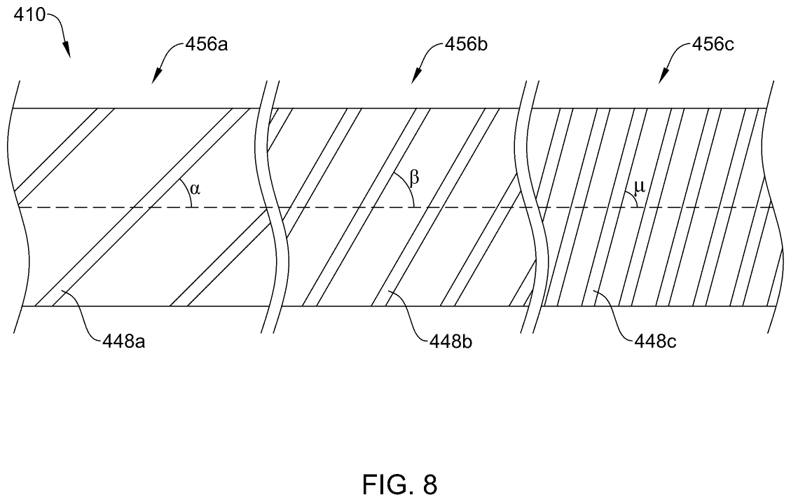

As indicated herein, variations in the properties of the catheter shafts disclosed herein may be primarily based on variation in the reinforcing layer. For example, FIG. 8 illustrates an example catheter shaft 410 with sections 456a, 456b, 456c. Each of the sections may include variability in the coil. For example, the coil section 448a in the catheter section 456a may have a greater coil spacing or pitch relative to the coil section 448b in the catheter section 456b. Stated another way, the coil section 448a in the catheter section 456a may have a lower surface area of coverage relative to the coil section 448b in the catheter section 456b. In addition, the coil section 448a may have a coil angle α that is smaller than a coil angle β along the coil section 448b. Likewise, the coil section 448b in the catheter section 456b may have a greater coil spacing or pitch (and/or a lower surface area of coverage) relative to the coil section 448c in the catheter section 456c. The coil angle β may be smaller than a coil angle μ along the coil section 448c. Other variations contemplated include variations in material of the coil where differing coil sections may vary in coil material.

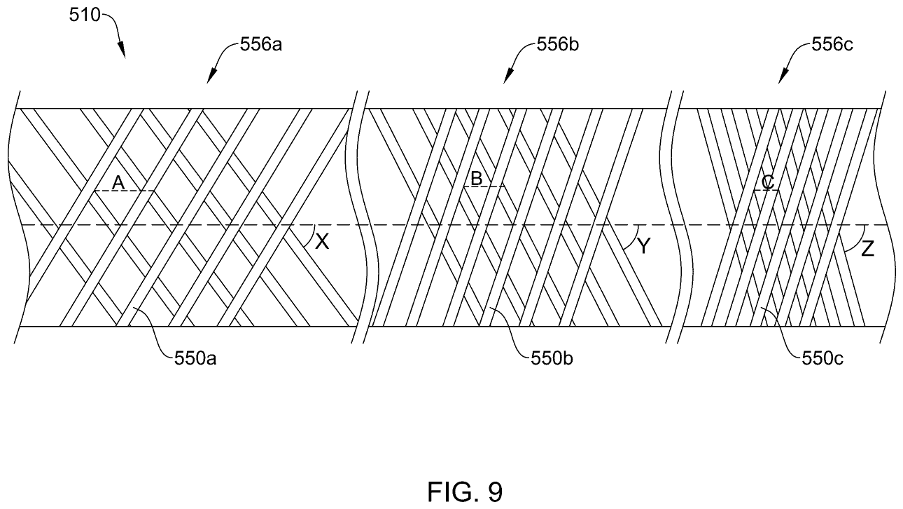

Similarly, FIG. 9 illustrates an example catheter shaft 510 with sections 556a, 556b, 556c. Each of the sections may include variability in the braid. For example, the braid section 550a in the catheter section 556a may have a braid spacing A that may be greater than a braid spacing B along the braid section 550b in the catheter section 556b. Likewise, the braid spacing B may be greater than a braid spacing C along the braid section 550c in the catheter section 556c. The braid angle X along the braid section 550a in the catheter section 556a may be smaller than the braid angle Y along the braid section 550b in the catheter section 556b. Likewise, the braid angle Y along the braid section 550b in the catheter section 556b may be smaller than the braid angle Z along the braid section 550c in the catheter section 556c. Other variations contemplated include variations in material of the braid where differing braid sections may vary in braid material. These are just examples. Variation and combination of different arrangements are contemplated.

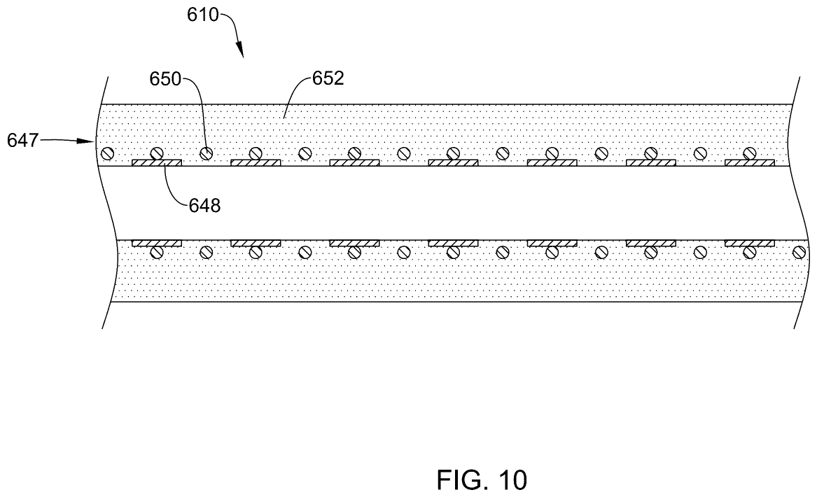

FIG. 10 illustrates another example catheter shaft 610 that is similar in form to other catheter shafts disclosed herein. The catheter shaft 610 may include a reinforcing layer 647 and an outer layer 652. In at least some instances, the reinforcing layer 647 may include a coil member 648, a braid 650, or both (e.g., as depicted). In this example, the catheter shaft 610 may lack a separate inner layer. Instead, the reinforcing layer 647 (e.g., the coil member 648) may form the innermost layer of the catheter shaft 610. In at least some instances, a portion of the outer layer 652 may migrate or extend through the reinforcing layer 647 and be disposed alongside the coil member 648 so as also to help define the innermost layer of the catheter shaft 610. This may help to maintain a smooth, continuous inner surface along the catheter shaft 610.

The materials that can be used for the various components of the systems disclosed herein may include those commonly associated with medical devices. For simplicity purposes, the following discussion refers to the catheter shaft 110. However, this is not intended to limit the devices and methods described herein, as the discussion may be applied to other similar shafts/sheaths disclosed herein.

The catheter shaft 110 may be made from a metal, metal alloy, polymer (some examples of which are disclosed below), a metal-polymer composite, ceramics, combinations thereof, and the like, or other suitable material. Some examples of suitable polymers may include ultra-high molecular weight polyethylene (UHMWPE), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), high-density polyethylene, low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some instances, the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

In at least some embodiments, portions or all of the catheter shaft 110 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the catheter shaft 110 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the catheter shaft 110 to achieve the same result.

In some instances, a degree of Magnetic Resonance Imaging (MRI) compatibility is imparted into the catheter shaft 110. For example, the catheter shaft 110, or portions thereof, may be made of a material that does not substantially distort the image and create substantial artifacts (e.g., gaps in the image). Certain ferromagnetic materials, for example, may not be suitable because they may create artifacts in an MRI image. The catheter shaft 110, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A catheter shaft, comprising:
a lubricious inner layer formed from a material that is free of per-and polyfluoroalkyl substances;
a support layer disposed over the lubricious inner layer, the support layer including a coil and a braid disposed over the coil; and
an outer layer extruded over the support layer.

2. The catheter shaft of claim 1, wherein the lubricious inner layer is formed from a continuous layer of a single material.

3. The catheter shaft of claim 1, wherein the lubricious inner layer includes ultra-high molecular weight polyethylene.

4. The catheter shaft of claim 1, wherein the coil comprises a single filar coil.

5. The catheter shaft of claim 1, wherein the coil comprises a ribbon coil.

6. The catheter shaft of claim 1, wherein the coil defines an area of coverage that varies along a length of the support layer.

7. The catheter shaft of claim 1, wherein the braid comprises a multi-strand braid.

8. The catheter shaft of claim 1, wherein the braid varies in braid angle along a length of the support layer.

9. The catheter shaft of claim 1, wherein the outer layer is formed from a continuous layer of a single material.

10. The catheter shaft of claim 1, wherein the outer layer is free of per- and polyfluoroalkyl substances.

11. The catheter shaft of claim 1, further comprising a steering member coupled to the catheter shaft.

12. The catheter shaft of claim 11, further comprising a steering wire coupled to the steering member and extending proximally therefrom.

13. The catheter shaft of claim 12, further comprising a steering lumen, wherein the steering wire extends at least partially through the steering lumen.

14. A method for manufacturing a catheter shaft, the method comprising:
disposing a lubricious material as a continuous lubricious inner layer along a mandrel, the continuous lubricious inner layer being formed from a material that is free of per- and polyfluoroalkyl substances;
disposing a support layer over the continuous lubricious inner layer, the support layer including a coil and a braid disposed over the coil; and
disposing an outer material as a continuous outer layer over the support layer.

15. The method of claim 14, wherein the lubricious material includes ultra-high molecular weight polyethylene.

16. The method of claim 14, wherein disposing a support layer over the continuous lubricious inner layer includes varying an area of coverage of the coil along a length of the support layer.

17. The method of claim 14, wherein disposing a support layer over the continuous lubricious inner layer includes varying a braid angle along a length of the support layer.

18. The method of claim 14, wherein disposing an outer material as a continuous outer layer over the support layer includes extruding the continuous outer layer over the support layer.

19. The method of claim 14, wherein the outer layer is free of per- and polyfluoroalkyl substances.

20. A catheter shaft, comprising:
a continuous lubricious inner layer formed from a material that is free of per- and polyfluoroalkyl substances;
a support layer disposed over the continuous lubricious inner layer, the support layer including a ribbon coil and a braid disposed over the ribbon coil; and
a continuous outer layer extruded over the support layer.
